(19) United States
(12) Patent Application Publication
(10) Pub. No.: US 2007/0190146 A1
(43) Pub. Date: Aug. 16, 2007

(54) Pharmaceutical Multilayer Tablet for Controlled Release of Active Ingredients With Highly pH-Dependent Solubility

(75) Inventors: Benedicte Roger, Paris (FR); Jean Luc Bruel, Bretigny Sur Orge (FR); Alain Cuine, Saint Fargeau-Ponthierry (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 11/622,118

(22) Filed: Jan. 11, 2007

Related U.S. Application Data
(63) Continuation of application No. PCT/EP05/08719, filed on Jul. 25, 2005.

(30) Foreign Application Priority Data
Jul. 29, 2004 (EP) 04291943.1

(51) Int. Cl. A61K 9/24

(52) U.S. Cl. 424/472

(57) ABSTRACT

The present invention relates to a pharmaceutical controlled release multilayer tablet comprising at least two layers, at least one active ingredient with highly pH-dependent solubility, at least one pharmaceutically acceptable pH maintaining excipient and at least one pharmaceutically acceptable matrix forming excipient, characterized in that said at least one active ingredient with highly pH-dependent solubility and said at least one pharmaceutically acceptable pH maintaining excipient are respectively comprised in at least one distinct layer.

The present invention relates to a novel pharmaceutical controlled release multilayer tablet, for controlled release of active ingredients with highly pH-dependent solubility.

### BACKGROUND OF THE INVENTION

Many active ingredients when formulated as immediate release conventional dosage forms, tablets, capsules, uncoated pellets, require administration several times each day. In such cases it is often advantageous to formulate the active ingredient as a controlled release formulation, so that the active ingredient is released gradually as it passes down the gastrointestinal tract, and is therefore absorbed slowly into the vascular system. The number of daily administrations may thus often be reduced, from three or four to two, and from two administrations to one. Such a form has the additional possible benefit that plasma levels of the active ingredient are often more constant than for immediate release forms, and so fewer side effects may be observed from excessively high peak levels just after dosing, and a better therapeutic cover is obtained.

A number of methods for achieving this slow and regular liberation from the dosage form are available to the person skilled in the art. Drug release may be slowed down by (i) slow diffusion through a membrane coating the dosage form, or by (ii) slow diffusion through a matrix, usually formed either by a polymer, or by a waxy substance or by a combination of both of these. The release rate in case (ii) may also be modulated by erosion of the dosage form, usually a matrix tablet, during its passage along the gastro-intestinal tract. Thus active ingredient release from such a matrix formulation may be by diffusion or erosion of the surface, or a combination of both of these.

A disadvantage often observed for the matrix tablet, whether a hydrophilic polymer or a lipidic excipient forms the matrix, is that the dissolution rate becomes slower with time. Release follows either a first order profile, and the rate decreases exponentially, or it follows the relationship first proposed by T. Higuchi, where the amount released is proportional to the square root of the time since release begun (Mechanism of Sustained-Action Medication: Theoretical Analysis of Rate of Release of Solid Drugs Dispersed in Solid Matrixes, J. Pharm. Sci. 12, 1145-9, 1963). In either case the rate decreases rapidly with time, whereas it would be advantageous for the rate to be constant.

Of the methods used to make the release rate more constant with time, one successful method has been perfected that consists in preparing tablets in several layers. One of the simplest forms is that where a tablet consists of three layers. The inner layer is a hydrophilic matrix comprising a cellulose derivative, and the active ingredient. The outer layers comprise hydrophilic polymers. The outer layers swell on contact with gastric and intestinal fluids and then erode. This erosion increases the surface of the inner layer exposed, facilitating liberation, and compensating for the slowing down of liberation with time normally observed for a matrix tablet.

A number of variations on this method have been described in U.S. Pat. No. 4,839,177, U.S. Pat. No. 5,422,123 and WO 98/08515. In another method disclosed in EP 0 598 309, a tablet can be formulated as two hydrophilic matrix disks comprising active ingredient, separated by an erodible disk, not comprising active ingredient. The outer layers swell to form matrices through which active ingredient diffuses slowly. Erosion of the central disk increases the exposed surface of the outer layers, until at last the tablet separates into two parts, with an increased surface and release rate, this again compensating for the normal slowing down of release from a matrix tablet.

Problems related to formulation of active ingredients with highly pH-dependent solubility within matrix tablets are constant and remain in multilayer tablets for the reasons explained hereinafter.

In particular, basic active ingredients, or salts thereof (i.e. salts of bases) have pH dependent solubilities, i.e. a solubility being low at pH 7 (neutral) but far higher under the acid conditions of the human stomach. Although they may be highly soluble at acid pH, many are slightly soluble or practically insoluble at neutral pH.

A classical formula related to apparent solubility of highly pH-dependent active ingredients, with a single basic group within the molecule, in relation to pH reads as follows:

$$S = {S}_{0} ( 1 + \frac{{10}^{pKa}}{{10}^{pH}} )$$

where S is the apparent solubility and S₀ is the solubility of the unprotonated base. The solubilities at pH 7 and pH 2 may differ by a factor of 10⁵. In addition, the solubility in media with pH 5.5 may be greater by up to 2 orders of magnitude than the solubility at pH 7,5, both values being commonly found in the small intestine and colon.

Acidic active ingredients may also exhibit highly pH dependent solubility. The solubility of the uncharged acid is often low at low pH, below the pKa of the acid, but it increases remarkably as the pH increases above the pKa. A formula corresponding to that given above for basic active ingredients relates the apparent solubility of acidic active ingredients, with a single acidic group within the molecule, in relation to pH as follows:

$$S = {S}_{0} ( 1 + \frac{{10}^{pH}}{{10}^{pKa}} )$$

where S is the apparent solubility and S₀ is the solubility of the undissociated acid.

Now, the rate of release from the dosage form depends on the solubility of the active ingredient at the local pH within the dosage form.

Since the matrix of the tablet must be permeable in order for the active ingredient to be released, its local pH within the dosage form (which we will call the “micro-pH”) will be influenced by the nature of the biological fluid surrounding it.

Moreover, a dosage form releases active ingredient into the biological fluids in the human gastrointestinal tract. A controlled slow release form may release active ingredient over a major part of the whole length of the gastrointestinal tract. The conditions for release are very different according to whether the dosage form is in the stomach, the small intestine or the colon and the pH of the medium surrounding the dosage form (which we will call the “external pH conditions”) will vary from acidic to neutral.

Thus, after the dosage form has been emptied from the stomach, the release of a basic active ingredient may slow down or almost stop, and so this simple method of obtaining a controlled release dosage form by incorporating an active ingredient with pH dependent solubility within a matrix fails in such cases. For the same reason the multilayer tablets, of the kind described by U. Conte, L. Maggi, P. Colombo, and A. La Manna, (Multi-layered hydrophilic matrixes as constant release devices (Geomatrix systems); J. Controlled Release 26:39-47 (1993)) fail to deliver a constant release rate independent of pH.

For this reason it is common, when formulating the active ingredient in a sustained release dosage form, to incorporate the active ingredient in the form of a salt, the rate of dissolution thus remaining constant whatever the pH. However, in the case of a basic active ingredient, basic ions can diffuse into the active ingredient dosage form from the intestinal fluid with the result that the micro-pH within the active ingredient dosage form is increased, and the free base precipitates. One way of overcoming this problem, and thus maintaining a constant release rate, is to add one or more acids, usually organic acids, or acid salts of polybasic organic acids to the active ingredient in the dosage form, in stoichiometric excess with respect to the active ingredient, to maintain a low pH within the dosage form. Thus the micro-pH within the active ingredient dosage form remains constant, and low. This approach is useful whether the basic active ingredient is incorporated in the dosage form as the free base, or as a salt. This has been done with simple matrix tablets, hydrophilic matrices (K. Ventouras and P. Buri, Role of the actification of hydrophilic matrices on the release of poorly soluble active substances in intestinal fluid, Pharm. Acta Helv., 52, 314-320 (1978)), wax matrices (WO 97/32584), and coated pellets (U.S. Pat. No. 5,616,345).

Similar effects are observed in the case of an acidic active ingredient formulated for sustained release. The acidic active ingredient may be released very slowly in the acidic conditions of the stomach, and then more rapidly after gastric emptying. If the acid active ingredient is incorporated as a salt, hydronium ions H₃O⁺ may diffuse into the dosage form from the gastric fluid, and cause the free acid to be precipitated within the dosage form. A base may be added to the dosage form to maintain a micro-pH higher than the pKa of the active ingredient.

An alternative approach in ensuring a micro-pH inside the dosage form independent of external pH conditions is to formulate an acidic active ingredient as the free acid, and to include an acid in the formulation. Similarly, a basic active ingredient may be formulated as the free base and a basic excipient added to the formulation. In this approach, the dissolution rate may be much slower.

In view of the above, a known method of ensuring release rate independent of pH, or of reducing the inhibitory effect of increasing pH on the release rate, for the multilayer tablets, is to add either a pharmaceutically acceptable acid or base, to the layer comprising either a basic or an acid active ingredient.

However, a first disadvantage of all these approaches is that frequently a large quantity of acid or base, to maintain the micro-pH, must be added. A second disadvantage is that pharmaceutically active ingredients are often chemically incompatible with acid or base in solid dosage forms.

More particularly, situations where it may be difficult using the prior art to formulate a basic or acidic active ingredient with highly pH-dependent solubility for controlled release are when one or more of the following characteristics are fulfilled:

(i) the solubility of the uncharged molecule of the active ingredient with highly pH-dependent solubility is less than 10 mg/l,

(ii) the total mass of active ingredient with highly pH-dependent solubility, within the multilayer tablet, is less than 20 mg,

(iii) the release of active ingredient with highly pH-dependent solubility is required to be over a period of above 8 hours,

(iv) the active ingredient with highly pH-dependent solubility is incompatible with strong acids, that is, for example, the presence of a strong acid provokes degradation of the active ingredient, or of a drug release-controlling excipient.

A considerable number of such active ingredients exist, and a high proportion of newly synthesized active ingredients are highly lipophilic and thus of low solubility at neutral pH. In addition, it is advantageous for the dose in active ingredient to be low, and for oral administration of the active ingredient to be administered once or at the most twice daily.

It has now been surprisingly found that a new dosage form may overcome the above problems in order to obtain a controlled release of basic or acidic active ingredients with highly pH-dependent solubility. In particular, the new dosage form according to the invention advantageously enables a constant micro-pH to be obtained, and a release rate whose dependence on the pH of the external medium is clearly reduced.

### SUMMARY OF THE INVENTION

Accordingly, the present invention relates to a pharmaceutical controlled release multilayer tablet comprising at least two layers, at least one active ingredient with highly pH-dependent solubility, at least one pharmaceutically acceptable pH maintaining excipient and at least one pharmaceutically acceptable matrix forming excipient, characterized in that said at least one active ingredient with highly pH-dependent solubility and said at least one pharmaceutically acceptable pH maintaining excipient are respectively comprised in at least one distinct layer.

### BRIEF DESCRIPTION OF THE DRAWINGS

FIG. 1 shows the percentage of active ingredient with highly pH-dependent solubility dissolved of the tablet described in example 2, as a function of time.

FIG. 2 shows the percentage of active ingredient with highly pH-dependent solubility dissolved of the tablet described in example 3, as a function of time.

FIG. 3 shows the percentage of active ingredient with highly pH-dependent solubility dissolved of the tablet described in example 4, as a function of time.

FIG. 4 shows the percentage of active ingredient with highly pH-dependent solubility dissolved of the tablet described in comparative example 1, as a function of time.

FIG. 5 shows the percentage of active ingredient with highly pH-dependent solubility dissolved of the tablet described in example 5, as a function of time.

FIG. 6 shows the percentage of active ingredient with highly pH-dependent solubility dissolved of the tablet described in comparative example 2, as a function of time.

FIG. 7 shows the percentage of active ingredient with highly pH-dependent solubility dissolved of the tablet described in example 6, as a function of time.

### DETAILED DESCRIPTION OF THE INVENTION

According to the present invention, “active ingredient with highly pH-dependent solubility” means any pharmaceutical active ingredient (basic or acidic) having respective solubilities, in a dissolution medium at pH 7 and in the same dissolution medium but at pH 2, which differ by a factor of at least 10, more particularly by a factor of at least 100.

By “distinct layer”, it should be understood that, according to a preferred embodiment of the present invention, there is essentially no pharmaceutically acceptable pH maintaining excipient in the layer(s) comprising said at least one active ingredient with highly pH-dependent solubility (being thus understood that any pharmaceutically acceptable pH maintaining excipient as defined below should not be present in a proportion exceeding 0.1% by weight, based on the total weight of the multilayer tablet, in the layer(s) comprising said at least one active ingredient with highly pH-dependent solubility) and, respectively, that there is essentially no active ingredient with highly pH-dependent solubility in the layer(s) comprising at least one pharmaceutically acceptable pH maintaining excipient (being thus understood that any active ingredient with highly pH-dependent solubility should not be present in a proportion exceeding 0.1% by weight, based on the total weight of active ingredient with highly pH-dependent solubility in the multilayer tablet, in the layer(s) comprising said at least one pharmaceutically acceptable pH maintaining excipient).

Furthermore, according to the present invention, “pH maintaining excipient” means any acid or acid salt thereof, and any base or basic salt thereof, known by the one skilled in the art, or a mixture thereof, adapted to obtain a constant micro-pH and a release rate whose dependence on the pH of the external medium is reduced. Depending on the desired rate of release, the pH maintaining excipient will either be acidic or basic, as explained above.

The pharmaceutical compositions according to the invention comprise a separate compartment of pH maintaining excipient. The embodiment according to the present invention consists in including the pH maintaining excipient in a separate layer or layers in a multilayer tablet. The present invention provides controlled release multilayer tablets characterized in that:
at least a first layer comprises an active ingredient with highly pH-dependent solubility with one or more excipients capable of forming a non disintegrating, swellable and/or erodible matrix, and additional excipients where necessary, acting as diluents, binders, lubricants and other tableting aids such as glidents;
at least a second layer is placed next to the first, comprising one or more pH maintaining excipient with excipients which can form a non-disintegrating swellable and/or erodible matrix. The excipients of the second layer (with the exception of the pH maintaining excipient) may be the same or different from those in the first layer.

Thus, in particular, the present invention relates to a pharmaceutical controlled release multilayer tablet, characterized in that it comprises:
at least one first type layer, comprising said at least one active ingredient with highly pH-dependent solubility and at least one pharmaceutically acceptable matrix forming excipient, and
at least one second type layer, placed next to said at least one first type layer, comprising said at least one pharmaceutically acceptable pH maintaining excipient and at least one pharmaceutically acceptable matrix forming excipient.

Thus, according to the above, the present invention more particularly relates to a pharmaceutical controlled release multilayer tablet comprising at least two layers, at least one active ingredient with highly pH-dependent solubility, at least one pharmaceutically acceptable pH maintaining excipient and at least one pharmaceutically acceptable matrix forming excipient, characterized in that said at least one active ingredient with highly pH-dependent solubility and said at least one pharmaceutically acceptable pH maintaining excipient are respectively comprised in at least one distinct layer, said pharmaceutical controlled release multilayer tablet comprising:
at least one first type layer, comprising said at least one active ingredient with highly pH-dependent solubility and at least one pharmaceutically acceptable matrix forming excipient, and
at least one second type layer, placed next to said at least one first type layer, comprising said at least one pharmaceutically pH maintaining excipient and at least one pharmaceutically acceptable matrix forming excipient.

As already above indicated, it should be understood that, according to a preferred embodiment of the present invention, there is essentially no pharmaceutically acceptable pH maintaining excipient in said at least one first type layer comprising said at least one active ingredient with highly pH-dependent solubility (being thus understood that any pharmaceutically pH maintaining excipient should not be present in a proportion exceeding 0.1% by weight, based on the total weight of the multilayer tablet, in said at least one first type layer comprising said at least one active ingredient with highly pH-dependent solubility) and, respectively, that there is essentially no active ingredient with highly pH-dependent solubility in said at least one second type layer comprising at least one pharmaceutically acceptable pH maintaining excipient (being thus understood that any active ingredient with highly pH-dependent solubility should not be present in a proportion exceeding 0.1% by weight, based on the total weight of active ingredient with highly pH-dependent solubility within the multilayer tablet, in said at least one second type layer comprising said at least one pharmaceutically acceptable pH maintaining excipient).

Thus, according to the above, the present invention more particularly relates to a pharmaceutical controlled release multilayer tablet comprising at least two layers, at least one active ingredient with highly pH-dependent solubility, at least one pharmaceutically acceptable pH maintaining excipient and at least one pharmaceutically acceptable matrix forming excipient, characterized in that said at least one active ingredient with highly pH-dependent solubility and said at least one pharmaceutically acceptable pH maintaining excipient are respectively comprised in at least one distinct layer, said pharmaceutical controlled release multilayer tablet comprising:
at least one first type layer, comprising said at least one active ingredient with highly pH-dependent solubility and at least one pharmaceutically acceptable matrix forming excipient, and
at least one second type layer, placed next to said at least one first type layer, comprising said at least one pharmaceutically pH maintaining excipient and at least one pharmaceutically acceptable matrix forming excipient, being understood that there is essentially no pharmaceutically acceptable pH maintaining excipient in said at least one first type layer comprising said at least one active ingredient with highly pH-dependent solubility and that there is essentially no active ingredient with highly pH-dependent solubility in said at least one second type layer comprising at least one pharmaceutically acceptable pH maintaining excipient.

Multilayer tablets with two layers: one of each type described above and with three layers: one in the middle of the first type and two of the second type placed up to the first, are preferred. In the multilayer tablets of three layers, the two outer layers of the second type may be identical in composition (qualitative and/or quantitative), or may differ from each other. Thus, in particular, the present invention relates to a pharmaceutical controlled release multilayer tablet characterized in that it consists of a two-layer tablet comprising:
one first type layer comprising said at least one active ingredient with highly pH-dependent solubility and at least one pharmaceutically acceptable matrix forming excipient, and
one second type layer, placed next to said first type layer, comprising said at least one pharmaceutically acceptable pH maintaining excipient and at least one pharmaceutically acceptable matrix forming excipient.

The present invention also relates in particular to a pharmaceutical controlled release multilayer tablet characterized in that it consists of a three-layer tablet comprising:
one first type layer comprising said at least one active ingredient with highly pH-dependent solubility and at least one pharmaceutically acceptable matrix forming excipient, and
two second type layers, placed next to said first type layer, each comprising said at least one pharmaceutically acceptable pH maintaining excipient and at least one pharmaceutically acceptable matrix forming excipient, these two second type layers being identical or not in composition (i.e. in qualitative and quantitative composition), said first type layer being placed between said two second type layers.

The present invention also relates in particular to a pharmaceutical controlled release multilayer tablet characterized in that it consists of a three-layer tablet comprising:
two first type layers, each comprising said at least one active ingredient with highly pH-dependent solubility and at least one pharmaceutically acceptable matrix forming excipient, these two first type layers being the same or not in composition (i.e. in qualitative and quantitative composition), and
one second type layer, placed next to said two first type layers, comprising said at least one pharmaceutically acceptable pH maintaining excipient and at least one pharmaceutically acceptable matrix forming excipient, said second type layer being placed between said two first type layers.

The said pharmaceutically acceptable pH maintaining excipient may be chosen among all pharmaceutically acceptable acids, acid salts thereof, and mixtures thereof, as well as among all pharmaceutically acceptable bases, basic salts thereof, and mixtures thereof, known by the person skilled in the art. In other words, said at least one pharmaceutically acceptable pH maintaining excipient is selected in the group consisting of pharmaceutically acceptable acids, acid salts thereof, and mixtures thereof, or in the group consisting of pharmaceutically acceptable bases, basic salts thereof, and mixtures thereof.

In particular, when said pH maintaining excipient is at least one pharmaceutically acceptable acid, acid salt thereof, or a mixture thereof, it is selected in the group consisting of organic acids, polybasic organic acids, inorganic acids, acid salts thereof, and mixtures thereof, and, when said pH maintaining excipient is at least one pharmaceutically acceptable base, basic salt thereof, or a mixture thereof, it is selected in the group consisting of organic bases, inorganic bases, basic salts thereof, basic salts of organic polybasic acids, basic salts of organic polybasic acids, and mixtures thereof.

More particularly, when said at least one pharmaceutically acceptable pH maintaining excipient is a pharmaceutically acceptable acid, acid salt thereof, or a mixture thereof, it has a pKa less than 6.5 and, when said at least one pharmaceutically acceptable pH maintaining excipient is a pharmaceutically acceptable base, basic salt thereof, or a mixture thereof, its conjugate acid has a pKa of greater than 7.5.

More particularly, when said pH maintaining excipient is at least one pharmaceutically acceptable acid or acid salt thereof, it is selected in the group consisting of tartaric acid, citric acid, succinic acid, fumaric acid, malic acid, malonic acid, adipic acid, gluconic acid, acid salts thereof, acid salts of phosphoric acid, and mixtures thereof, and, when said pH maintaining excipient is at least one pharmaceutically acceptable base or basic salt thereof, it is selected in the group consisting of trisodium phosphate, tripotassium phosphate, calcium carbonate, basic salts of pyrophosphoric acid, sodium carbonate, magnesium carbonate, magnesium oxide, magnesium aluminosilicate, and mixtures thereof.

The new dosage form according to the present invention enables an excess of pH maintaining excipient to be used, being at least 10% by weight, based on the total weight of the tablet, and a physical separation of pH maintaining excipient and active ingredient during manufacturing and storage, right up to the time of ingestion.

In particular, the proportion of said at least one pH maintaining excipient is comprised between 5 and 50% by weight, and more particularly between 8 and 25% by weight, based on the total weight of the multilayer tablet.

According to the present invention, “pharmaceutically acceptable matrix forming excipient” means any pharmaceutically acceptable excipient capable of forming a non disintegrating swellable and/or erodible matrix in a matrix tablet, as well known by the person skilled in the art.

In particular, said at least one pharmaceutically acceptable matrix forming excipient is selected in the group consisting of hydrophilic polymers, amphiphilic polymers, lipidic excipients and mixtures thereof.

More particularly, said at least one pharmaceutically acceptable matrix forming excipient is selected in the group consisting of hydroxypropylmethylcellulose (or “hypromellose”), hydroxypropylcellulose, hydroxyethylcellulose, methylcellulose, ethylcellulose, polymethacrylates (including methacrylate copolymers), polyoxyethylene, polyacrylic acid, polyvinyl acetate, polyoxyethylene-polyoxypropylene copolymer, hydrogenated castor oil, carnauba wax, and mixtures thereof.

According to the present invention, said at least one pharmaceutically acceptable matrix forming excipient may be the same or different in each first type and second type layer of the multilayer tablet.

As a particular technical advantage of the present invention, it is possible to use a pharmaceutically acceptable matrix forming excipient that is unstable and/or incompatible to acids in the layer(s) comprising the active ingredient with highly pH-dependent solubility. Indeed, certain matrix forming excipients used to control release of the active ingredient are unstable to acid, and thus the release profile may change over a period of time when a tablet comprising such a matrix forming substance is in contact with an acid. In particular, because of acid catalysed hydrolysis of matrix forming polymeric excipient into lower molecular weight fragments, the drug release profile can become faster, and the drug dosage form no longer control release of the drug. Examples of matrix forming substances unstable to acids are derivatives of cellulose, in particular hydroxypropylmethylcellulose, hydroxypropylcellulose, hydroxyethylcellulose methylcellulose and ethylcellulose.

Thus, as a particular embodiment of the present invention, said at least one pharmaceutically acceptable matrix forming excipient of said first type layer is selected in the group consisting of hydroxypropylmethylcellulose, hydroxypropylcellulose, hydroxyethylcellulose, methylcellulose, ethylcellulose, polymethacrylates, polyoxyethylene, polyvinylacetate, polyacrylic acid, polyoxyethylene-polyoxypropylene copolymer, hydrogenated castor oil, carnauba wax, and mixtures thereof, and said at least one pharmaceutically acceptable matrix forming excipient of said second type layer is selected in the group consisting of polymethacrylates (including methacrylate copolymers), polyoxyethylene, polyvinylacetate, polyacrylic acid, polyoxyethylene-polyoxypropylene copolymer, hydrogenated castor oil, carnauba wax, and mixtures thereof.

Of course, as well known by the person skilled in the art, the multilayer tablet of the present invention may further comprise at least one pharmaceutically acceptable excipient selected in the group consisting of diluents, binders, water-channelling agents, lubricants, glidents, and mixtures thereof. Examples of such possible additional excipients are summarized in the following table.

TABLE 1

| Excipient | |
|---|---|
| function | Possible excipients for the first and second type layers |
| Diluents | lactose, mannitol, microcrystalline cellulose, calcium |
| | hydrogen phosphate, tricalcium phosphate, pregelatinised |
| | starch, cross-linked starch |
| Binders | Hydroxypropylmethylcellulose, methylcellulose, povidone, |
| | polyvinyl alcohol |
| Water- | Crospovidone, sodium carboxymethylcellulose, sodium |
| channelling | starch glycolate |
| agents | |
| Lubricants | Stearic acid and its alkaline earth salts, sodium stearyl |
| and | fumarate, glyceryl behenate, colloidal silicon dioxide, |
| glidants | talc |

As it will be understood by the person skilled in the art, each layer of the multilayer tablet according to the present invention may comprise one or more of such additional excipients above cited. These excipients and others with the same or additional functions will be combined together as is known to the person skilled in the art to give the desired release profile in a dissolution test

According to the present invention said at least one active ingredient with highly pH-dependent solubility is a basic one or an acidic one.

In particular, said at least one active ingredient with highly pH-dependent solubility presents at least one of the following characteristics:

(i) the solubility of the uncharged molecule of the active ingredient with highly pH-dependent solubility is less than 10 mg/l,

(ii) the total mass of active ingredient with highly pH-dependent solubility, within the multilayer, tablet is less than 20 mg,

(iii) the release of the active ingredient with highly pH-dependent solubility is required to be over a period of above 8 hours,

(iv) the active ingredient with highly pH-dependent solubility is incompatible with strong acids, that is, for example, the presence of a strong acid provokes degradation of the active ingredient, or of a drug release-controlling excipient.

More particularly, said at least one active ingredient with highly pH-dependent solubility is selected in the group consisting of N-[2-[[4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)₃-[1-(2-phenylethyl)piperidin-4-yl]-1,3,4-oxodiazol-2(3H)-one hydrochloride, 7-fluoro-2-oxo-4-[2-[4(thieno[3,2-c]pyridin-4-yl)piperazin-1-yl]ethyl]-1,2-dihydroquinoline-1-acetamide, clopidogrel, mizolastin, pravastatin, naproxen, acetylsalicylic acid, diclofenac, zolpidem, and salts thereof.

According to the present invention, the proportion of said active ingredient with highly pH-dependent solubility is comprised between 0.1 and 30% by weight, more particularly between 0.5 and 15% by weight, based on the total weight of the multilayer tablet. The multilayer tablet according to the present invention may thus comprise, for example, from 0.1 to 100 mg of active ingredient with highly pH-dependent solubility.

The multilayer tablet according to the present invention may be prepared following methods well known by the person skilled in the art. For example, it can be prepared in two steps: different powders are first manufactured corresponding to the first type or the second type layer composition, as described above, and the compressed to form the multilayer tablet. The powders may be simple mixtures and the tablet formed by direct compression. Alternately, the mixture of excipients for the first type or second type layer may be granulated, according to one or other of the methods of granulation commonly known by the person skilled in the art of pharmaceutical formulation: granulation with water or another liquid, dry granulation, hot melt granulation.

These granulates may eventually be coated with a protecting polymer or lipid coating chosen among ethylcellulose, polymethacrylates, polyacrylic acid, hydrogenated castor oil, carnauba wax in order to control the release rate.

After preparation of the two kinds of powders by granulation or by simple mixing, they are compressed to give layered tablets consisting of two or more layers in a multilayer tableting machine.

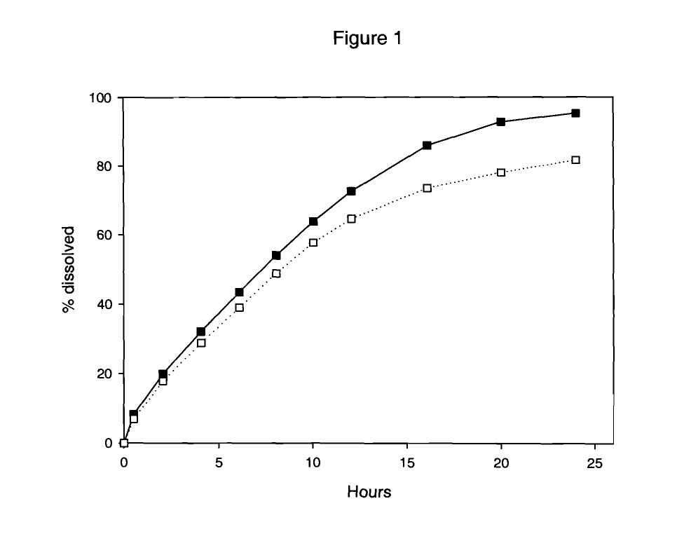

In FIGS. 1-7, the full line (filled black squares or filled black circles) shows dissolution in 0.01 M hydrochloric acid (pH 2), and the dotted line (empty squares or empty circles) shows dissolution in a 0.006 M potassium phosphate buffer (pH 6.8).

FIG. 1 shows the percentage of active ingredient with highly pH-dependent solubility dissolved of the tablet described in example 2, as a function of time.

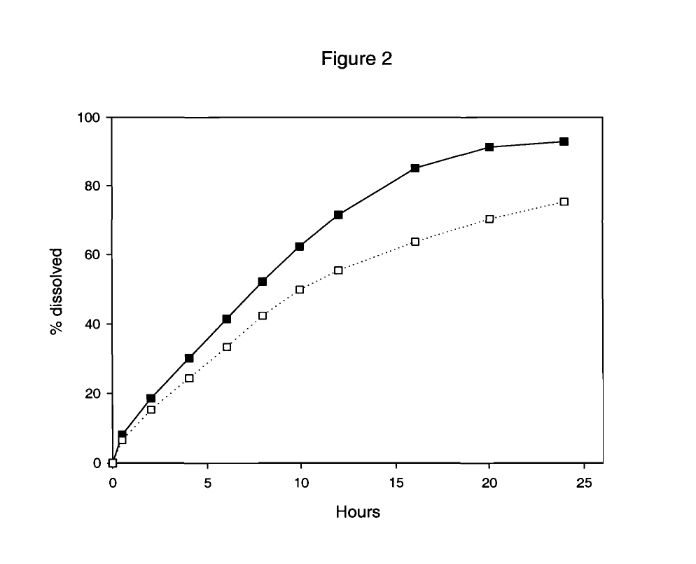

FIG. 2 shows the percentage of active ingredient with highly pH-dependent solubility dissolved of the tablet described in example 3, as a function of time.

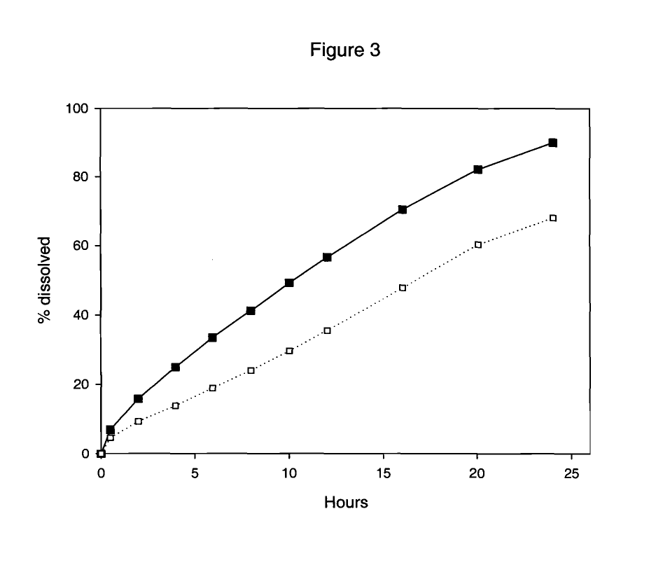

FIG. 3 shows the percentage of active ingredient with highly pH-dependent solubility dissolved of the tablet described in example 4, as a function of time.

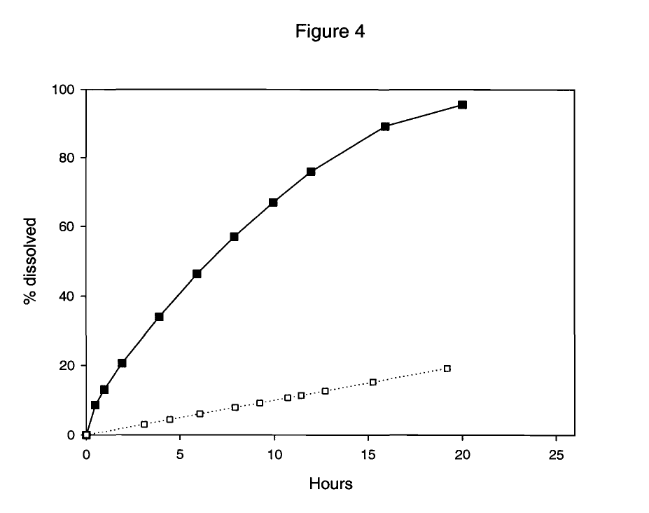

FIG. 4 shows the percentage of active ingredient with highly pH-dependent solubility dissolved of the tablet described in comparative example 1, as a function of time.

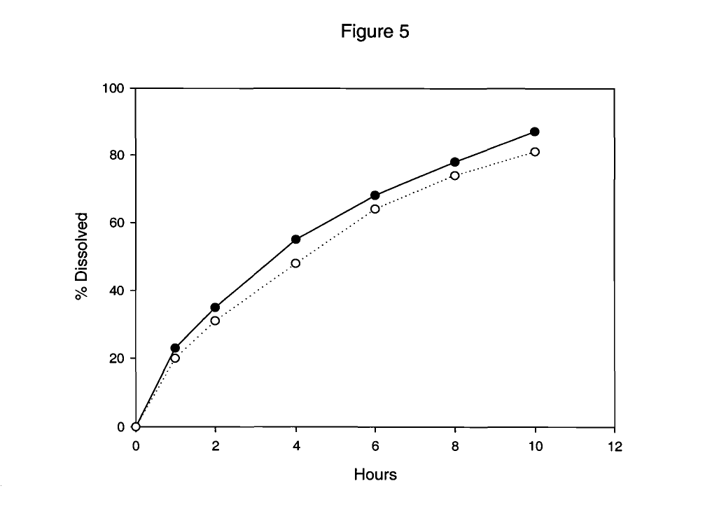

FIG. 5 shows the percentage of active ingredient with highly pH-dependent solubility dissolved of the tablet described in example 5, as a function of time.

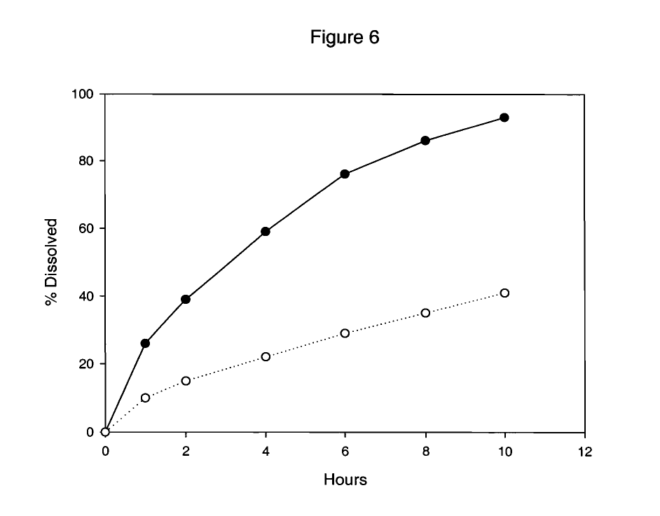

FIG. 6 shows the percentage of active ingredient with highly pH-dependent solubility dissolved of the tablet described in comparative example 2, as a function of time.

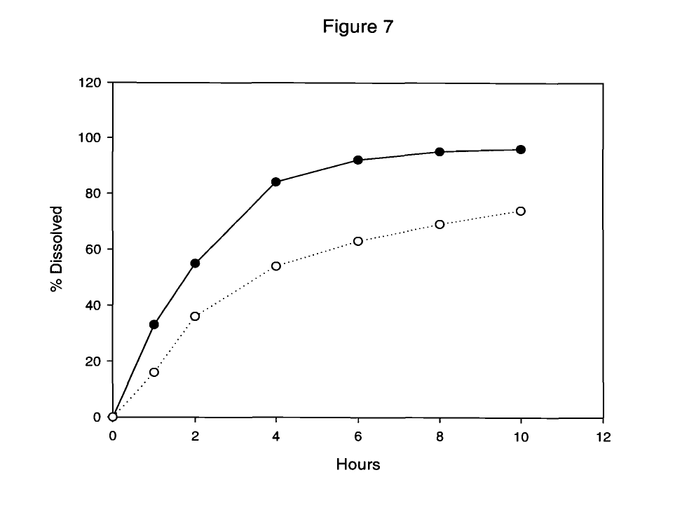

FIG. 7 shows the percentage of active ingredient with highly pH-dependent solubility dissolved of the tablet described in example 6, as a function of time.

The following examples are intended to illustrate the present invention and should thus not be construed as limiting the scope of the present invention.

In the following examples, some were performed with a active ingredient described in example 1 of EP 577 470 chemically named N-[2-[[4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, in the form of its methanesulfonate salt useful in the treatment of benign prostatic hyperplasia, hereinafter called “Drug 1”.

### EXAMPLE 1

### Granulate Comprising Drug 1 and Hydroxypropylmethylcellulose

A granulate A was prepared from the following mixture (except magnesium stearate and Aerosil), by aqueous granulation using a Hobart mixer-granulator. The granulate was then dried in an oven at 50° C., calibrated to 0.8 mm, then lubricated by mixing in the remaining constituents.

| | |
|---|---|
| Drug 1 | 11.6% |
| Hydroxypropylmethylcellulose (Methocel ® K100M) | 10.0% |
| Mannitol 60 | 20.0% |
| Microcrystalline cellulose (Avicel ® PH101) | 54.0% |
| Povidone K29/32 | 3.2% |
| Colloidal silicon dioxide (Aerosil ® 200) | 0.2% |
| Magnesium stearate | 1.0% |
| | 100.0% |

### EXAMPLE 2

### Three-Layer Tablet with Succinic Acid in the Outer Layers

A granulate B was prepared comprising succinic acid, as follows. The method was the same as for example 1.

| | |
|---|---|
| Hydroxypropylmethylcellulose (Methocel ® K100M) | 35.0% |
| Lactose 150M | 24.5% |
| Microcrystalline cellulose (Avicel ® PH101) | 13.9% |
| Succinic acid | 20.0% |
| Povidone K29/32 | 5.0% |
| Iron oxide (yellow) | 0.4% |
| Colloidal silicon dioxide (Aerosil ® 200) | 0.2% |
| Magnesium stearate | 1.0% |
| | 100.0% |

Three-layer tablets were manufactured with the granulate A from example 1 as the inner layer, dosed at 11.6 mg of Drug 1 and the above granulate B comprising acid for the two outer layers. Each layer contained 100 mg of granulate. The compression was carried out using an alternating tableting machine Frogerais AO, using size 8R16 punches. Each layer (100 mg for each layer) was filled manually. The in vitro dissolution was then tested at pH 2 and pH 6.8, using the following method.

The apparatus described in the European Pharmacopoeia was used. Agitation was by the paddle method (100 rpm). The dissolution medium was continuously sampled by means of a peristaltic pump, and the UV absorbance measured by a double beam UV spectrophotometer. The percentage of Drug 1 dissolved was determined at each measured time point by comparison with the absorbance of a standard solution of 11.6 μg.ml⁻¹ Drug 1 in the dissolution medium. The dissolution medium was 500 ml of 0.01 M hydrochloric acid or 500 ml potassium phosphate buffer, pH 6.8, 0.006 M. Results are shown in FIG. 1.

### EXAMPLE 3

### Three-Layer Tablet with Tartaric Acid in the Outer Layers

A granulate C was prepared in exactly the same way as the granulate B of example 2, and with the same composition except tartaric acid was used instead of succinic acid. Three-layer tablets using granulate A comprising Drug 1 for the inner layer and granulate C (with tartaric acid) for the outer layers were prepared as in example 2. Their in vitro dissolution was then tested at pH 2 and pH 6.8, using the same dissolution method as in example 2.

Results are shown in FIG. 2.

### EXAMPLE 4

### Three-Layer Tablet with Fumaric Acid in the Outer Layers

A granulate D was prepared in exactly the same way as the granulate B of example 2, and with the same composition except that fumaric acid was used instead of succinic acid. Three-layer tablets using granulate A comprising Drug 1 in the inner layer and granulate D (comprising fumaric acid) for the outer layers were prepared as in example 2. Their in vitro dissolution was then tested at pH 2 and pH 6.8, using the same dissolution method as in example 2, except that the results were corrected for the UV absorbance of fumaric acid by subtracting the profile obtained by dissolution of a placebo tablet. Results are shown in FIG. 3.

### COMPARATIVE EXAMPLE 1

### Three-Layer Tablet Without Acid

A granulate E was prepared in exactly the same way as the granulate B of example 2, with the following composition:

| | |
|---|---|
| Hydroxypropylmethylcellulose (Methocel ® K100M) | 35.0% |
| Lactose 150M | 34.5% |
| Microcrystalline cellulose (Avicel ® PH101) | 23.9% |
| Povidone K29/32 | 5.0% |
| Iron oxide (yellow) | 0.4% |
| Colloidal silicon dioxide (Aerosil ® 200) | 0.2% |
| Magnesium stearate | 1.0% |
| | 100.0% |

Three-layer tablets using granulate A comprising Drug 1 for the inner layer and granulate E (without acid) for the outer layers were prepared as in example 2. Their in vitro dissolution was then tested at pH 2 and pH 6.8, using the same dissolution method as in example 2. Results are shown in FIG. 4: it can be seen that the dissolution is very similar to that of the tablet comprising acid at pH 2 (example 2, FIG. 1), but very much slower at neutral pH.

These examples show that various acids are adapted to multilayer tablets, as pH maintaining excipient, to obtain profiles of dissolution wherein rates tend to be constant whatever the pH of the dissolution medium.

A stability study showed improved results with the tablet of the above example 2 in comparison with a single layer tablet i.e. a tablet comprising said Drug 1 and succinic acid in the same single layer. In particular, the tablet of example 2 did not show any non-acceptable yellow colouring after a 13 weeks storage, while this was the case with the single layer tablet, deemed as a consequence of a compatibility problem between said Drug 1 and succinic acid.

### EXAMPLE 5

### Three-Layer Tablet with two Outer Layers Containing Tartaric Acid and an Inner Layer Containing Zolpidem Tartrate

A granulate G not containing active ingredient but containing hypromellose and tartaric acid was prepared using the same process as for the granulate B of example 2, according to the composition:

| | |
|---|---|
| Tartaric acid | 12.0% |
| Hydroxypropylmethylcellulose | 28.0% |
| (or “Hypromellose”; Metholose ®90SH4000SR) | |
| Lactose 150 mesh | 38.8% |
| Microcrystalline cellulose (Avicel ® PH101) | 20.0% |
| Colloidal silicon dioxide (Aerosil ® 200) | 0.2% |
| Magnesium stearate | 1.0% |
| | 100.0% |

A granulate H containing zolpidem tartrate, was prepared with the same process according to the composition:

| | |
|---|---|
| Zolpidem tartrate | 5.0% |
| Hydroxypropylmethylcellulose | 12.0% |
| (or “Hypromellose”; Metholose ®90SH4000SR) | |
| Lactose 150 mesh | 61.8% |
| Microcrystalline cellulose (Avicel ® PH101) | 20.0% |
| Colloidal silicon dioxide (Aerosil ® 200) | 0.2% |
| Magnesium stearate | 1.0% |
| | 100.0% |

Three-layer tablets using granulate H for the inner layer and granulate G for the outer layers were prepared as in example 2. Their in vitro dissolution was then tested at pH 2 and pH 6.8, using the following method.
The apparatus described in the European Pharmacopoeia was used. Agitation was by the paddle method (100 rpm). The dissolution medium was continuously sampled by means of a peristaltic pump, and the UV absorbance measured by a UV spectrophotometer. The percentage of zolpidem tartrate dissolved was determined at each measured time point by comparison with the absorbance of a standard solution of 10.0 μg.ml⁻¹ zolpidem tartrate in the dissolution medium. The dissolution medium was 500 ml of 0.01 M hydrochloric acid or 500 ml potassium phosphate buffer, pH 6.8, 0.015 M. The results are shown in FIG. 5.

### COMPARATIVE EXAMPLE 2

### Three-Layer Tablet with Two Outer Layers Without Acid and an Inner Layer Containing Zolpidem Tartrate

A granulate I containing hypromellose, but neither active substance nor acid was prepared in the same way as the granulate B of example 2, according to the composition:

| | |
|---|---|
| Hydroxypropylmethylcellulose | 28.0% |
| (or “Hypromellose”; Metholose ®90SH4000SR) | |
| Lactose 150 mesh | 50.8% |
| Microcrystalline cellulose (Avicel ® PH101) | 20.0% |
| Colloidal silicon dioxide (Aerosil ® 200) | 0.2% |
| Magnesium stearate | 1.0% |
| | 100.0% |

Three layer tablets using granulate H containing the zolpidem tartrate for the inner layer and granulate I (without acid) for the outer layers were prepared as in example II. Their in vitro dissolution was then tested at pH 2 and pH 6,8, using the same dissolution method as in example IV. Results are shown in FIG. 6.

### EXAMPLE 6

### Two-Layer Tablet with a Layer Containing Tartaric Acid and Methacrylate Copolymer and a Second Layer Containing Zolpidem Tartrate

A granulate J without active ingredient but containing tartaric acid and methacrylate copolymer was prepared in the same way as the granulate B of example 2, according to the composition:

| | |
|---|---|
| Tartaric acid | 12.0% |
| Methacrylate copolymer (Eudragit NE40D) | 12.0% |
| Lactose 150 mesh | 54.8% |
| Microcrystalline cellulose (Avicel ® PH101) | 20.0% |
| Colloidal silicon dioxide (Aerosil ® 200) | 0.2% |
| Magnesium stearate | 1.0% |
| | 100.0% |

A granulate K containing zolpidem tartrate and hypromellose, was prepared in the same way as the granulate A, according to the composition:

| | |
|---|---|
| Zolpidem tartrate | 5.0% |
| Hydroxypropylmethylcellulose | 28.0% |
| (or “Hypromellose”; Metholose ®90SH4000SR) | |
| Lactose 150 mesh | 45.8% |
| Microcrystalline cellulose (Avicel ® PH101) | 20.0% |
| Colloidal silicon dioxide (Aerosil ® 200) | 0.2% |
| Magnesium stearate | 1.0% |
| | 100.0% |

Two-layer tablets using granulate K containing the product for the first layer and granulate J for the second layer were prepared as in example 2. Their in vitro dissolution was then tested at pH 2 and pH 6.8, using the same dissolution method as in example 5. Results are shown in FIG. 7.

What is claimed is:

1. A pharmaceutical controlled release multilayer tablet comprising at least two layers, at least one active ingredient with highly pH-dependent solubility, at least one pharmaceutically acceptable pH maintaining excipient and at least one pharmaceutically acceptable matrix forming excipient, wherein said at least one active ingredient with highly pH-dependent solubility and said at least one pharmaceutically acceptable pH maintaining excipient are respectively comprised in at least one distinct layer.

2. A pharmaceutical controlled release multilayer tablet according to claim 1, comprising:
at least one first type layer, comprising said at least one active ingredient with highly pH-dependent solubility and at least one pharmaceutically acceptable matrix forming excipient; and
at least one second type layer, placed next to said at least one first type layer, comprising said at least one pharmaceutically acceptable pH maintaining excipient and at least one pharmaceutically acceptable matrix forming excipient.

3. A pharmaceutical controlled release multilayer tablet according to claim 1, consisting of a two-layer tablet comprising:
one first type layer comprising said at least one active ingredient with highly pH-dependent solubility and at least one pharmaceutically acceptable matrix forming excipient; and
one second type layer, placed next to said first type layer, comprising said at least one pharmaceutically acceptable pH maintaining excipient and at least one pharmaceutically acceptable matrix forming excipient.

4. A pharmaceutical controlled release multilayer tablet according to claim 1, consisting of a three-layer tablet comprising:
one first type layer comprising said at least one active ingredient with highly pH-dependent solubility and at least one pharmaceutically acceptable matrix forming excipient; and
two second type layers, placed next to said first type layer, each comprising said at least one pharmaceutically acceptable pH maintaining excipient and at least one pharmaceutically acceptable matrix forming excipient, these two second type layers being identical or not in composition, said first type layer being placed between said two second type layers.

5. A pharmaceutical controlled release multilayer tablet according to claim 1, consisting of a three-layer tablet comprising:
two first type layers, each comprising said at least one active ingredient with highly pH-dependent solubility and at least one pharmaceutically acceptable matrix forming excipient, these two first type layers being the same or not in composition; and
one second type layer, placed next to said two first type layers, comprising said at least one pharmaceutically acceptable pH maintaining excipient and at least one pharmaceutically acceptable matrix forming excipient, said second type layer being placed between said two first type layers.

6. A pharmaceutical controlled release multilayer tablet according to claim 1, wherein at least one said pharmaceutically acceptable pH maintaining excipient is selected from the group consisting of pharmaceutically acceptable acids, acid salts thereof, and mixtures thereof.

7. A pharmaceutical controlled release multilayer tablet according to claim 1, wherein at least one said pharmaceutically acceptable pH maintaining excipient is selected from the group consisting of pharmaceutically acceptable bases, basic salts thereof, and mixtures thereof.

8. A pharmaceutical controlled release multilayer tablet according to claim 6, wherein at least one said pharmaceutically acceptable pH maintaining excipient is selected from the group consisting of organic acids, polybasic organic acids, inorganic acids, acid salts thereof, and mixtures thereof.

9. A pharmaceutical controlled release multilayer tablet according to claim 7, wherein at least one said pharmaceutically acceptable pH maintaining excipient is selected in the group consisting of organic bases, inorganic bases, basic salts thereof, basic salts of organic polybasic acids, basic salts of organic polybasic acids, and mixtures thereof.

10. A pharmaceutical controlled release multilayer tablet according to claim 6 wherein, where said at least one pharmaceutically acceptable pH maintaining excipient is a pharmaceutically acceptable acid, acid salt thereof, or a mixture thereof, it has a pKa less than 6.5.

11. A pharmaceutical controlled release multilayer tablet according to claim 7 wherein, where said at least one pharmaceutically acceptable pH maintaining excipient is a pharmaceutically acceptable base, basic salt thereof, or a mixture thereof, its conjugate acid has a pKa of greater than 7.5.

12. A pharmaceutical controlled release multilayer tablet according to claim 1, wherein at least one said pharmaceutically acceptable pH maintaining excipient is selected from the group consisting of tartaric acid, citric acid, succinic acid, fumaric acid, adipic acid, malic acid, malonic acid, gluconic acid, acid salts thereof, acid salts of phosphoric acid, and mixtures thereof.

13. A pharmaceutical controlled release multilayer tablet according to claim 1, wherein at least one said pharmaceutically acceptable pH maintaining excipient is selected from the group consisting of trisodium phosphate, tripotassium phosphate, calcium carbonate, basic salts of pyrophosphoric acid, sodium carbonate, magnesium carbonate, magnesium oxide, magnesium aluminosilicate, and mixtures thereof.

14. A pharmaceutical controlled release multilayer tablet according to claim 1, wherein the proportion of said at least one pharmaceutically acceptable pH maintaining excipient is between 5 and 50% by weight, based on the total weight of the multilayer tablet.

15. A pharmaceutical controlled release multilayer tablet according to claim 1, wherein said at least one pharmaceutically acceptable matrix forming excipient is selected from the group consisting of hydrophilic polymers, amphiphilic polymers, lipidic excipients and mixtures thereof.

16. A pharmaceutical controlled release multilayer tablet according to claim 15, wherein said at least one pharmaceutically acceptable matrix forming excipient is selected from the group consisting of hydroxypropylmethylcellulose, hydroxypropylcellulose, hydroxyethylcellulose, methylcellulose, ethylcellulose, polymethacrylates, polyoxyethylene, polyacrylic acid, polyvinyl acetate, polyoxyethylene-polyoxypropylene copolymer, hydrogenated castor oil, carnauba wax, and mixtures thereof.

17. A pharmaceutical controlled release multilayer tablet according to claim 2, wherein said at least one pharmaceutically acceptable matrix forming excipient may be the same or different in each first type and second type layer.

18. A pharmaceutical controlled release multilayer tablet according to claim 2, characterized in that said at least one pharmaceutically acceptable matrix forming excipient of said first type layer is selected from the group consisting of hydroxypropylmethylcellulose, hydroxypropylcellulose, hydroxyethylcellulose, methylcellulose, ethylcellulose, polymethacrylates, polyoxyethylene, polyvinyl acetate, polyacrylic acid, polyoxyethylene-polyoxypropylene copolymer, hydrogenated castor oil, carnauba wax, and mixtures thereof; and said at least one pharmaceutically acceptable matrix forming excipient of said second type layer is selected from the group consisting of polymethacrylates, polyoxyethylene, polyvinyl acetate, polyacrylic acid, polyoxyethylene-polyoxypropylene copolymer, hydrogenated castor oil, carnauba wax, and mixtures thereof.

19. A pharmaceutical controlled release multilayer tablet according to claim 1, further comprising at least one pharmaceutically acceptable excipient selected from the group consisting of diluents, binders, water-channelling agents, lubricants, glidents and mixtures thereof.

20. A pharmaceutical controlled release multilayer tablet according to claim 1 wherein said at least one active ingredient with highly pH-dependent solubility is a basic one.

21. A pharmaceutical controlled release multilayer tablet according to claim 1, wherein said at least one active ingredient with highly pH-dependent solubility is an acidic one.

22. A pharmaceutical controlled release multilayer tablet according to claim 1, wherein said at least one active ingredient with highly pH-dependent solubility presents at least one characteristic selected from:
the solubility of the uncharged molecule of the active ingredient with highly pH-dependent solubility is less than 10 mg/l;
the total mass of active ingredient with highly pH-dependent solubility, within the multilayer, tablet is less than 20 mg;
the release of the active ingredient with highly pH-dependent solubility is required to be over a period of above 8 hours; and
the active ingredient with highly pH-dependent solubility is incompatible with strong acids.

23. A pharmaceutical controlled release multilayer tablet according to claim 1, wherein said at least one active ingredient with highly pH-dependent solubility is selected from the group consisting of N-[2-[[4-aminocarbonyl)pyrimidin-2-yl]amino]ethyl]-2-[[3-[4-(5-chloro-2-methoxyphenyl)piperazin-1-yl]propyl]amino]pyrimidine-4-carboximide, 5-(8-amino-7-chloro-2,3-dihydro-1,4-benzodioxin-5-yl)₃-[1-(2-phenylethyl)piperidin-4-yl]-1,3,4-oxodiazol-2(3H)-one hydrochloride, 7-fluoro-2-oxo-4-[2-[4(thieno[3,2-c]pyridin-4-yl)piperazin-1-yl]ethyl]-1,2-dihydroquinoline-1-acetamide, clopidogrel, mizolastin, pravastatin, naproxen, acetylsalicylic acid, diclofenac, zolpidem, and salts thereof.

24. A pharmaceutical controlled release multilayer tablet according to claim 1, wherein the proportion of said at least one active ingredient with highly pH-dependent solubility is between 0.1 and 30% by weight, based on the total weight of the multilayer tablet.
